(19) United States
(12) Patent Application Publication
(10) Pub. No.: US 2018/0028068 A1
(43) Pub. Date: Feb. 1, 2018

(54) METHOD AND SYSTEM FOR NONINVASlVELY MONITORING CONDITIONS OF A SUBJECT

(71) Applicant: OR-NIM MEDICAL LTD., Kfar Saba (IL)

(72) Inventors: Zeev SCHIFFER, Petach Tikva (IL); Eliahu RATNER, Jerusalem (IL); Ilan BRESKIN, Tel-Aviv (IL); Adi TSALACH, Ramat Hasharon (IL); Avihai RON, Kfar Saba (IL)

(21) Appl. No.: 15/548,888

(22) PCT Filed: Feb. 3, 2016
(86) PCT No.: PCT/IL16/50122

§ 371 Date: Aug. 4, 2017

Related U.S. Application Data
(60) Provisional application No. 62/112,366, filed on Feb. 5, 2015.
(60) Provisional application No. 62/112,381, filed on Feb. 5, 2015.

(51) Int. Cl. A61B 5/00

(52) U.S. Cl. CPC A61B 5/0097; A61B 5/7257; A61B 5/725; A61B 2576/00

(57) ABSTRACT

A measurement system for determining at least one parameter of a subject, the system comprising: an acoustic device for generating and irradiating a region of interest in the subject with acoustic tagging radiation having a carrier frequency and being modulated by a predetermined coding function of at least one parameter varying over time; an optical device for illuminating the region of interest with electromagnetic radiation of a predetermined frequency range, detecting an electromagnetic radiation response of the region of interest, and generating measured data indicative of interaction between the acoustic tagging radiation and the electromagnetic radiation at successive positions in the region of interest, the optical device being operable concurrently with the acoustic device during at least a first measurement session; and a control unit for processing the measured data and determining at least first data comprising spectral data as a function of position, such that each of the measured successive positions in the region of interest is characterized by its spectral data.

### TECHNOLOGICAL FIELD AND BACKGROUND

This invention is generally in the field of medical devices, and relates to a method and system for monitoring subject's conditions, based on ultrasound tagging of light. The invention is particularly useful for characterizing the media/tissues and identifying or locating and/or measuring a parameter of flow in a flow-containing medium in a region of interest in tissues, such as brain, muscle, kidney and other organs.

Non invasive monitoring and imaging using non-ionizing radiation allows medical professionals to diagnose and monitor a patient condition without invasive procedures, e.g. eliminating a need for drawing blood. Some of the non-invasive monitoring methods rely on monitoring the optical properties of a tissue by illuminating the tissue and detecting a light response of the tissue. If the tissue is homogenous, simple models allow for the calculation of optical properties. However, as biological tissues are complex scattering media, measuring the local optical properties becomes a challenging task.

WO 2008/149342, assigned to the assignee of the present invention, discloses a method and system for use in determining one or more parameters of a subject. According to this technique, a region of interest of the subject is irradiated with acoustic tagging radiation, and at least a portion of the region of interest is concurrently irradiated with electromagnetic radiation of a predetermined frequency range. Electromagnetic radiation response of the at least portion of the region of interest is detected, and measured data indicative thereof is generated, where the detected response comprises electromagnetic radiation tagged by the acoustic radiation. The measured data indicative of the detected electromagnetic radiation response is processed to determine at least one parameter of the subject in a region corresponding to the locations in the medium at which the electromagnetic radiation has been tagged by the acoustic radiation.

### GENERAL DESCRIPTION

There is a need in the art to provide a novel measurement technique enabling to fully characterize a tissue at different depths of a region of interest, without losing or at least significantly reducing losses of information about the optical properties of the tissue at each and every depth.

There is also a need in the art to provide a novel measurement technique that provides accurate measurement on a subject regardless of the condition of the subject being measured, as well as the environment of the measurement procedure and the depth of a region of interest being examined inside the subject. For example, there is a need that the measurements performed on two subjects would potentially have the same medical/physical meaning and can be compared. In addition, two measurement procedures performed on the same subject at different times or in different environments should be identical for the same measured medical condition. Moreover, it is desirable to obtain an online indication for the measurement quality that may enable carrying out required actions to ensure an adequate measurement quality.

The present invention utilizes “Ultrasound Tagging of Light” (UTL) which is an effect based on the interaction of acoustic waves with the same tissue volume that is being probed by light. This interaction causes the light wave to be modulated, or tagged, with the characteristics of the acoustic wave (i.e. frequency, phase). As the propagation of acoustic waves in tissue is relatively slow (about 1500 m/sec in soft tissue), the location of the interaction of light with the acoustic radiation can be determined. The signal obtained by taking only the carrier frequency component of the acoustic radiation calculated for each delay, is termed here carrier frequency ultrasound tagged light (CFUTL), and is identical to the cross correlation between the coded signal used to generate the transmitted acoustic (ultrasound) wave (also termed the coding function) and the detected light signal, as has been previously described in WO 2008/149342, assigned to the assignee of the present application.

The efficiency and power of the interaction of the acoustic waves with the medium affects the spatial and temporal resolution and the Signal to Noise Ratio (SNR) of the measurement. There are three possible modalities for the generation of acoustic waves, a continuous wave (CW), a short burst of waves (SB), and a pulse. Operation with continuous waves produces a higher SNR, because more acoustic energy is irradiated and detected. When a continuous acoustic wave (at a predetermined frequency range) interacts with light, and light is collected throughout the full propagation of the acoustic waves, a higher acoustic energy is available for the interaction, thereby increasing the signal. In addition, the spectral bandwidth of the continuous acoustic wave can be very narrow, thus reducing noise bandwidth. Thereby the SNR is greatly improved. However, the spatial resolution of a measurement produced with continuous acoustic waves is not as high as a measurement produced with short bursts or pulses of acoustic waves. This reduced spatial resolution is particularly limiting when the measurement geometry calls for propagation of acoustic waves essentially parallel to the direction of light propagation. As for the use of short bursts of waves and pulses, this provides better spatial resolution, but the acoustic energy of the interaction is lower and the bandwidth is wider as compared to those of a continuous wave mode, resulting in reduced SNR. In order to achieve both high spatial resolution and high SNR, the inventors have introduced a method, disclosed in WO 2008/149342, that utilizes generation of continuous acoustic waves (and therefore improving the SNR), where the continuous acoustic wave is a modulated (coded) signal characterized by a narrow autocorrelation function, thereby improving the spatial resolution.

To achieve the above mentioned goals of the measurement technique, adequate coupling of the acoustic and optical radiations to the examined tissue should be guaranteed, but even in case an optimal coupling could not be achieved, a calibration/normalization of the measured data may be acquired to compensate for the less optimal coupling conditions. In addition, it is desirable to indicate sub-optimal coupling conditions so that appropriate action could be taken (e.g. improvement of the coupling during the measurement or applying a different downstream processing method to the data, either on-line or offline). As the coupling conditions deteriorate and the UTL signal levels are reduced, often the noise levels are not reduced by the same amount and the SNR is also reduced, such that even if the coupling influence on the average measurement value is compensated for, the overall measurement quality is reduced and the ability to extract significant information from the data is compromised.

According to the present invention, a sample volume is irradiated with a modulated acoustic (ultrasound) wave, of a certain carrier frequency usually, using a specifically generated coded signal; and is concurrently illuminated by electromagnetic radiation of a predetermined wavelength range, such that ultrasound and light interact in successive volumes (positions, depths) of the tissue along an axis of the ultrasound propagation. Light backscattered from the tissue is detected, this detected light includes tagged light shifted to a frequency range centered at the carrier frequency of ultrasound, as well as untagged light. The detected light signal is analyzed both in the time and frequency domains, and a delay-frequency distribution is obtained. The delay is usually a function of the distance (depth) along the ultrasound propagation axis. The detected light comprises data portions indicative of light returned from multiple depths in the tissue. This detected light is decoded, such that an independent signal is obtained for each delay (depth) separately.

Spectral domain analysis (e.g. Fourier transform, spectral filtering, etc.) of each such decoded time-trace signal enables extracting depth-specific spectral-domain parameters (e.g. spectral peak width, amplitude, etc.), and information relating to flow/movement of optical scattering centers within the sample/tissue, at that specific depth. The obtained parameters may be accumulative, such as spectral width at a certain delay, or differential, obtained by comparing (e.g. by subtracting, dividing, or other mathematical operations) the parameter obtained for one delay with the parameter obtained for a second delay. More generally, the obtained parameters may be a result of applying a mathematical operation on parameters obtained for one or more delays, additional examples including a linear combination and a non-linear combination.

By using the depth-specific spectral domain processing results, it is possible to deduce physical parameters regarding the mapped sample. These physical parameters may be, but not limited to, the optical de-correlation time as a function of depth, the distribution of flow vs. depth in absolute units, the calibrated distribution of flow vs. depth in units of flow, or the acoustic coupling quality. One of the important possible parameters is the blood-oxygen saturation level, which may be obtained by using a pulsed coded acoustic radiation.

A flow of fluid within the sampled volume (e.g. blood flow) increases movement of scattering objects leading to increased variability in the phase accumulated along the different propagation paths. The width of the power spectrum peaks of the detected light backscattered from the sample at a frequency range around the acoustic carrier frequency is affected by frequency broadening effects, such as Doppler broadening due to motion of scattering centers within the monitored medium of the sample. As flow increases, the amplitude of the detected light at the ultrasound frequency decreases, while the width of the spectral component containing the ultrasound frequency increases (assuming other conditions remain unchanged). The power spectrum profile is therefore indicative of flow parameters within the sample.

According to the present invention, a spectrum for each delay comprising multiple frequencies may be calculated using the detected signal of the first measurement session, thus each volume/location is characterized by its spectral data. It should be understood that a specific delay corresponds to a specific measured location, being a location of interaction between ultrasound, tissue and light. The present invention provides for sifting the accumulative spectral broadening and extracting the local contribution of the movement of depth-specific scattering centers, to the total power spectrum.

As already has been said, when a sample is irradiated concurrently with ultrasound (generally, acoustic radiation) and electromagnetic radiation, the resulting spectrum of the detected electromagnetic radiation response of the sample is affected by photons from all the depths, and particularly from those traveling in shallower depths, as they are statistically much more probable to arrive at the detector. The spectrum is actually a weighted sum of spectra donated by photons propagating in different paths. In order to observe frequency changes caused by specific layers (volumes) in the sample, it is possible to excite by ultrasound only a specifically given depth (localized layer/volume), and data indicative of light returned from/tagged at this specific depth could be extracted and discriminated. This localized excitation (“Tagging”) can be done, for example, by modulating ultrasound amplitude with a narrow pulse shape (narrow in the time-domain) so that only a specific layer is spatially excited at a given time. As ultrasound propagates through the tissue, different depths are radiated with corresponding time delays of the ultrasound radiation. Hence, different time delays yield spectra which correspond to different depths in the sample. Yet, the spectral width associated with a specific depth will be composed of incremental donations of all intermediate layers within that distance from the ultrasound transmission plane. Spectral broadening generated at a given depth (local broadening) may be deduced by differentiating spectral widths of adjacent layers (adjacent time delays). Changes in spectral width are attributed to location of flow, while the amount of broadening is related to volumetric flow rate.

An alternative to the localized excitation with a temporally narrow pulse shape, is to excite the tissue continuously (i.e. long pulses with >100 excitation cycles) with a coded excitation function, followed by decoding the measured signal such that tagging events occurring at different locations in the tissue are separated to different signals that can be processed and analyzed separately. One advantage of this technique is that it enables to transmit more energy to the tissue, resulting in a larger signal that enables reliable extraction of information.

The UTL signal depends on the amplitude of light and the amplitude of the acoustic pressure wave that is coupled to the tissue. Thus, in order to determine the optical properties of the tissue, such as frequency/color (oxygen saturation) and local blood flow effects, there is a need to decouple the two parameters (light and acoustic energy).

The decoupling of the ultrasound may be achieved by using several wavelengths of light, and dividing the UTL profiles obtained for each wavelength one by the other. This is described in WO 2008/149342. However, when only one wavelength of light is used, decoupling the effect of variability in the amplitude of the ultrasound waves that are coupled into the tissue, on the obtained UTL light profile, may be achieved in another way.

The invention provides a technique for determining optical properties of a tissue, e.g. characteristic de-correlation time, by potentially eliminating the ultrasound coupling effect on the detected light signal. This allows for calculating a depth-flow distribution or a calibrated blood flow parameter (calibrated Calculated Flow Index, cCFI) being potentially independent of the ultrasound coupling, for example by dividing the spectral peak amplitude of the UTL by the energy of light parameter in a spectral band around the carrier frequency (of the acoustic radiation) computed in one specific depth (a scalar), or by the energy of light in a spectral band around the carrier frequency computed and averaged from multiple depths (a scalar), or by the total energy of light being the sum of light energies in a spectral band around the carrier frequency computed at all depths (a scalar) or by the energy of light in a spectral band around the carrier frequency computed for each depth (a vector, an element-wise division).

The division of the UTL by any options described above, or others, or using the inverse term of any of these calculations, mitigates the undesired effects of the variability of the optical and acoustic coupling conditions on the UTL, allowing obtaining “absolute units” of depth-flow distribution or a calibrated depth-flow distribution.

As said, the energy of light parameter for each delay (depth), also termed local light energy parameter, is obtained by integrating the power spectrum calculated at that delay along the frequency axis with a certain bandwidth (BW) around the ultrasound carrier frequency. Similarly, the overall light energy is the sum of power at a certain bandwidth (BW) around the ultrasound carrier frequency, calculated for all the power spectra at all delays.

It should be noted that the term “light energy” refers to a certain predetermined function of spectral data, and should thus be interpreted broadly and be not limited to the mathematical meaning of energy, i.e. squared light intensity.

The present invention provides a novel technique for improving the accuracy of the UTL based measurements. This is done by normalizing the detected light signal formed by light tagged by acoustic radiation. This detected signal is referred to herein as “UTL signal”. The present invention also provides a means to assess the acoustic coupling and indicate the measurement quality. The normalization provides that the UTL signal associated with a certain measurement location in the region of interest is not influenced by the variability of the optical and acoustic signal amplitude associated with conditions external to the subject, such as the light source output power, the acoustic source output power, the optical coupling conditions, the acoustic coupling conditions and so on. Additionally, the inventors also found how to extract and use extra spectral data extracted from the detected light radiation from each depth in the region of interest.

According to the invention, the subject (region of interest) may undergo two measurement sessions. Generally, the two measurement sessions may be performed concurrently using two different light detectors, e.g. by using a different carrier frequency for the acoustic radiation in each measurement session; or successively, in either order, using similar or different carrier frequencies for the acoustic radiation. It should be understood, that the terms “first” and “second” used herein do not mean that the first precedes the second, but are used only to distinguish between the two measurement sessions which can be run, as mentioned above, either simultaneously or sequentially in either order. One of the measurement sessions operates with irradiating the region with coded acoustic radiation (for example coded with a Golay code) of a certain (first) carrier frequency, detection of the light intensity signal including ultrasound tagged and untagged light, and calculation of the intensity of ultrasound tagged light which is frequency-shifted by the carrier frequency of ultrasound as a function of position (depth) according to the acoustic radiation delay. The second measurement session operates with CW uncoded acoustic radiation of a certain (second) carrier frequency which be identical or different from the first carrier frequency, detection of the light intensity signal including tagged & untagged light, and computing the total tagged light energy, which is the energy of detected light in a predetermined frequency range around the carrier frequency. At the processing stage, the signal detected in the first measurement session is normalized by dividing the tagged light position function (UTL) by the total tagged light energy acquired in the second measurement session. The normalization step mitigates the undesired effects of the external optical and acoustic conditions, e.g. coupling conditions, on the UTL, allowing to obtaining absolute-unit flow index or a calibrated flow measurement. The total tagged light energy is also used to assess acoustic coupling condition and indicate the measurement quality.

Thus according to a broad aspect of the present invention, there is provided a measurement system for use in determining at least one parameter of a subject, said system comprising:

(a) an acoustic device adapted for generating acoustic tagging radiation and for irradiating a region of interest of the subject with said acoustic tagging radiation propagating with a general propagation direction, said acoustic tagging radiation comprising modulated acoustic radiation in the form of acoustic wave having a carrier frequency and being modulated by a predetermined coding function of at least one parameter of the acoustic tagging radiation varying over time;

(b) an optical device adapted for illuminating the region of interest with electromagnetic radiation of a predetermined frequency range, detecting an electromagnetic radiation response of the region of interest, and generating measured data corresponding to the detected electromagnetic radiation response; said optical device being operable concurrently with the acoustic device during at least a first measurement session, the measured data being thereby indicative of the electromagnetic radiation response to interaction between the acoustic tagging radiation and the electromagnetic radiation at successive positions in the region of interest along said general propagation direction during said at least first measurement session, corresponding to successive delays of the interaction between the acoustic tagging radiation and the electromagnetic radiation during said at least first measurement session, and

(c) a control unit adapted for processing the measured data and determining at least first data comprising spectral data as a function of position within the region of interest along said general propagation direction of the acoustic tagging radiation through the region of interest, such that each of the measured successive positions in the region of interest is characterized by its spectral data.

In some embodiments, the present invention concerns modulation of ultrasound waves obtained using a Golay code as the predetermined function.

In some embodiments, the processing of the measured data comprises: multiplying the measured data by an envelope of said predetermined function (e.g. the Golay code) shifted at different delays, the product of multiplication by each delay being indicative of the electromagnetic radiation response arriving from a portion/location of the region of interest corresponding to said delay; and performing spectral processing (e.g. a Fourier transform) on the product of multiplication by the different delays, thereby obtaining a spectral broadening parameter for each delay (depth).

In some embodiments, the processing of the measured data comprises: multiplying the measured data by an envelope of said predetermined function (e.g. the Golay code) shifted at different delays, the product of multiplication by each delay being indicative of the electromagnetic radiation response arriving from a portion/location of the region of interest corresponding to said delay; and applying at least one spectral domain filter on the product of multiplication by the different delays, thereby obtaining a spectral broadening parameter for each delay (depth).

In some embodiments, the processing of the measured data comprises applying spectral analysis to spectral data from the successive positions along the trajectory of propagation of the electromagnetic radiation, thereby determining localized spectral broadening data of specific positions. The spectral analysis may comprise determining a linear combination of the spectral data from the successive positions along the trajectory of propagation of the electromagnetic radiation. The spectral analysis may comprise subtracting spectral data of first and second successive positions along the trajectory of propagation of the electromagnetic radiation, thereby determining localized spectral broadening data of the second position.

In some embodiments, the processing of the measured data further comprises calculating a flow-depth distribution with absolute units. The calculating may comprise determining a parameter of a profile of the spectral data in one or more of the successive positions along the trajectory of propagation of the electromagnetic radiation. The calculating may comprise determining a width parameter of at least one peak in the spectral data in one or more of the successive positions along the trajectory of propagation of the electromagnetic radiation. At times, the calculating comprises dividing a light energy parameter of the detected electromagnetic radiation by amplitude of a cross correlation between the coding function of the tagging acoustic radiation and the detected electromagnetic radiation signal.

In some embodiments, the light energy parameter comprises the light energy in a spectral band around the carrier frequency in one specific position in the region of interest. In some embodiments, the light energy parameter comprises an average of light energies in a spectral band around the carrier frequency in a plurality of positions in the region of interest. In some embodiments, the light energy parameter comprises a vector of light energies in a spectral band around the carrier frequency at least two positions in the region of interest.

In some embodiments, the processing of the measured data further comprises calculating a calibrated Calculated Flow Index (cCFI), being a function of the spectral data. The calculation may comprise determining a width parameter of at least one peak in the spectral data in one or more of the successive positions along the trajectory of propagation of the electromagnetic radiation. The calculation may comprise dividing a total energy parameter of the detected electromagnetic radiation by amplitude of a cross correlation between the coding function of the tagging acoustic radiation and the detected electromagnetic radiation. According to some embodiment, the processing comprises obtaining a local energy parameter for each delay by integrating power spectrum calculated at that delay along a frequency axis, and determining the total energy parameter as a sum of all the local energy parameters.

In some embodiments, the processing of the measured data comprises calculating a carrier frequency ultrasound tagged light (CFUTL) signal as a cross correlation between said predetermined coding function of at least one parameter and said electromagnetic radiation response.

In some embodiments, the acoustic device is further adapted for generating acoustic tagging radiation in the form of a continuous uncoded acoustic wave having a second carrier frequency, which may be identical or different than the first carrier frequency, to propagate along said general propagation direction, thereby causing interaction between the continuous acoustic radiation and the electromagnetic radiation at the region of interest, said measured data further comprising data indicative of detected electromagnetic radiation response from the region of interest to said interaction with the continuous acoustic radiation; said control unit being adapted for processing said measured data and determining second data comprising spectral data of the region of interest, and utilizing at least one of the first and second data for determining a total energy parameter of the tagged portion of the detected electromagnetic radiation in a predetermined frequency range around the second carrier frequency. The first and second carrier frequencies may be identical or different.

In some embodiments, the first and second data are obtained during first and second successive measurement sessions, which may or may not be of equal time intervals.

In some embodiments, the processing of the first measured data comprises calculating a carrier frequency ultrasound tagged light (CFUTL) signal. The processing may further comprise dividing the CFUTL signal by the total energy parameter.

In some embodiments, the processing of the second measured data comprises calculation of the spectral width of the second measured data.

In some embodiments, the processing of either one of the first and second measured data comprises determining Fourier transform of the data.

In some embodiments, the spectral processing of either one of the first and second measured data comprises applying spectral filtering to the data.

In some embodiments, the determination of total energy parameter comprises obtaining a local energy parameter for each delay by integrating power spectrum calculated at that delay along a frequency axis, and determining the total energy parameter as a sum of all local energy parameters.

According to another broad aspect, there is provided a system for use in determining one or more parameters of a subject, said system comprising;

(a) an optical device configured for illuminating a region of interest with electromagnetic radiation of a predetermined frequency range, and for detecting an electromagnetic radiation response from said region of interest, and for generating measured data indicative of the detected electromagnetic radiation response;

(b) an acoustic device configured for irradiating said region of interest, while being illuminated, with first and second acoustic radiations propagating with a general propagation direction during respective first and second measurement sessions, wherein: the first acoustic radiation comprises acoustic tagging radiation in the form of acoustic wave having a first carrier frequency and modulated by a predetermined coding function of at least one parameter of the first acoustic tagging radiation varying over time, the second acoustic radiation comprising acoustic tagging radiation in the form of a continuous uncoded acoustic wave having a second carrier frequency, the measured data thereby comprising first and second data indicative of first and second interactions between the electromagnetic radiation with respectively first and second acoustic tagging radiations within the region of interest and the electromagnetic radiation at successive positions of the region of interest during the first and second measurement sessions; and

(c) a control unit configured and operable to process the first and second data, said processing comprising: determining first spectral data indicative of first electromagnetic radiation response from successive positions of the region of interest corresponding to successive delays of the interaction between the first acoustic tagging radiation and the electromagnetic radiation during said first measurement session, and second spectral data indicative of second electromagnetic radiation response of the region of interest and a total energy parameter of tagged portion of electromagnetic radiation around the second carrier frequency.

According to yet further aspect, the invention provides a monitoring system for use in determining one or more parameters of a subject, the monitoring system comprising a control unit comprising:

a data input utility configured for receiving measured data comprising at least first data indicative of ultrasound tagged light of interaction between coded acoustic tagging radiation of a first carrier frequency and electromagnetic radiation of a predetermined frequency range at successive locations along an acoustic radiation propagation axis within a region of interest corresponding to successive delays of the interaction during at least first measurement session time interval; and

a data processor and analyzer configured for analyzing the measured data and determining spectral data of acoustically tagged electromagnetic radiation as a function of position within the region of interest along said general propagation axis, such that each of successive positions in the region of interest is characterized by its spectral data.

The control unit is configured for data communication with a measurement unit which generates the measured data, and/or a storage device where the measured data is stored. The measurement unit is configured for generating acoustic tagging radiation having a carrier frequency, being in the form of an acoustic wave modulated by a predetermined coding function of at least one parameter of the acoustic radiation varying over time, and generating light of a predetermined frequency range, and for detecting light of this frequency range comprising the ultrasound tagged light, and generating the measured data. In the case when two measurement sessions are performed as described above, the control unit is also configured for generating acoustic tagging radiation having a carrier frequency in the form of unmodulated acoustic wave.

The processor and analyzer utility comprises: a first processing module configured for processing the first measured data to obtain delay-distribution data, a second processing module configured for calculating the total tagged light energy and a third processing module configured for calculating the normalization of the delay-distribution data obtained by the first module, by the total tagged light energy obtained by the second module. The delay-distribution data obtained from the first processing module may be one-dimensional, having a single value per each depth, or 2-dimensional, having a plurality of values per each depth. Non-limiting examples of one-dimensional delay-distribution data are the CFUTL signal, and a signal that contains a spectral-width value per each depth. An example of two-dimensional data is the delay-frequency distribution obtained by calculating a power spectrum signal per each depth. Thus, the first processing module may comprises a decoder module configured for multiplying the measured data by an envelope of a coding function shifted at different delays, and a second spectral-processing module configured for performing spectral processing on a product of multiplication, e.g. applying a Fourier transform or filtering techniques, thereby obtaining a delay-frequency distribution data indicative of a position spectral data through the region of interest along the axis of progression, being indicative of at least one parameter of the region of interest. Alternatively, the first processing module may comprise a module for calculation of a cross-correlation between the coding function and the measured light intensity signal, yielding the CFUTL.

The second processing module that calculates the total tagged light energy from the second measurement session featuring an uncoded CW acoustic signal is configured to extract, from the uncoded UTL signal, energy from a predetermined bandwidth around the carrier frequency. Obtaining the total tagged light energy may be done, for example, by applying a spectral band-pass filter on the UTL signal followed by applying an integrator that integrates the filtered signal power to obtain the total energy. Another way for such extraction of the total tagged light energy is to apply a Fourier transform and to calculate the uncoded UTL's power spectrum, followed by applying an integrator that integrates the power in the frequency domain to obtain the total energy.

The third processing module is configured to receive a first processed data indicative of the delay-distribution data from the first processing module and a second processed data indicative of the total tagged light energy from the second processing module, and dividing the first processed data by the second processed data thereby obtaining third processed data indicative of normalized delay-distribution data.

The processor and analyzer utility may comprise a cross-correlation module configured for calculating cross-correlation between the predetermined coding function and the first measured data, thereby obtaining correlated data indicative of intensity of the tagged light in the first measured data arriving from successive locations along the propagation axis in the region of interest, the correlated data being indicative of at least one parameter of the region of interest.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a schematic illustration of an example of a measurement system according to the present invention;

FIG. 1B is a flow diagram exemplifying a method of the invention carried out by the system of FIG. 1A for obtaining a 2D delay-frequency distribution,

FIG. 1C is a flow diagram exemplifying another method of the invention carried out by the system of FIG. 1A,

FIG. 2A is a graphical representation exemplifying a delay-frequency distribution as a whole, and cross sections along specific frequency and specific delay (depth),

FIGS. 2B and 2C illustrate broadening effects of the spectrum in connection with depth and ultrasound excitation location,

FIGS. 3A and 3B present results obtained from a liquid phantom in which the liquid contains stirred scattering centers, where FIG. 3B shows the power spectrum obtained at three different depths (distances/delays) from the ultrasound wave source,

FIGS. 4A and 4B show a schematic diagram of a liquid channel phantom used for creating and recording different signals from different depths (FIG. 4A), and the local spectral broadening effect at the different depths (FIG. 4B),

FIG. 5 illustrates an experimental setup used to simulate a state of flow and a state of no-flow conditions in a liquid phantom,

FIG. 6 shows the power spectrum and energy of tagged electromagnetic radiation obtained in a flow and in a no-flow conditions,

FIG. 7 illustrates the linear relationship between the mean energy of tagged light and the ultrasound amplitude,

FIGS. 8 and 9 illustrate the effect of changing the electromagnetic illumination intensity on the detected tagged electromagnetic radiation energy, while keeping the acoustic radiation constant,

FIGS. 10 and 11 illustrate the effect of changing the amplitude of the acoustic tagging radiation on the detected electromagnetic radiation energy, while keeping the electromagnetic illumination intensity constant, and

FIG. 12 exemplifies the difference in the relation between the flow index (FI) calculation and the acoustic radiation amplitude, when calculating the FI is based on UTL normalization using the electromagnetic radiation energy or on UTL normalization using the total energy parameter.

### DETAILED DESCRIPTION OF EMBODIMENTS

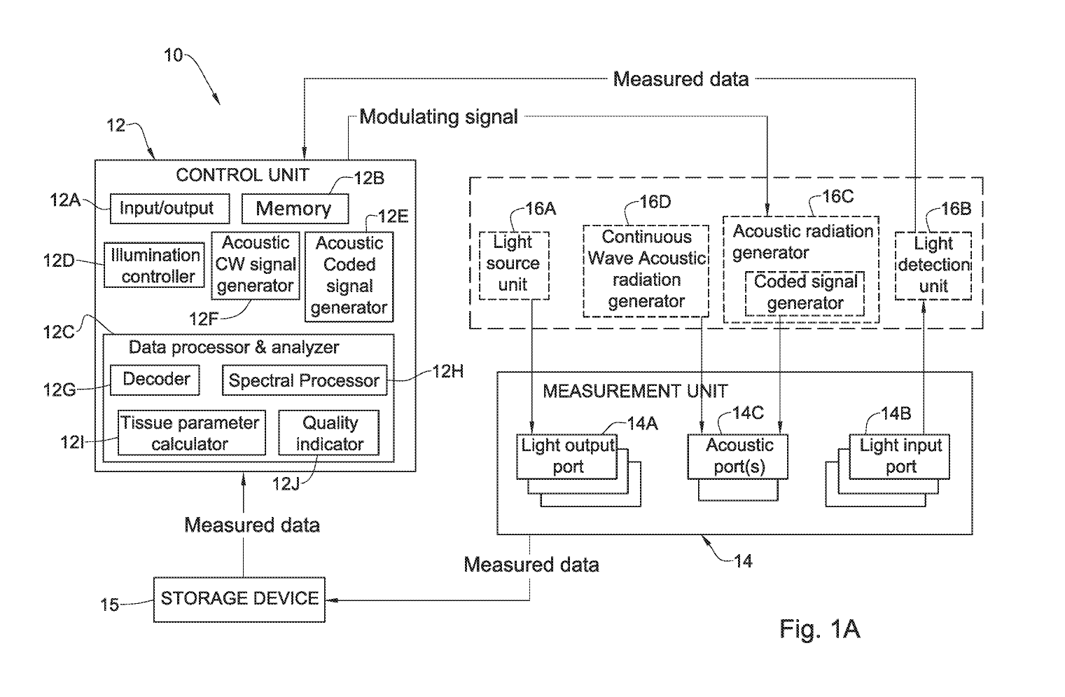

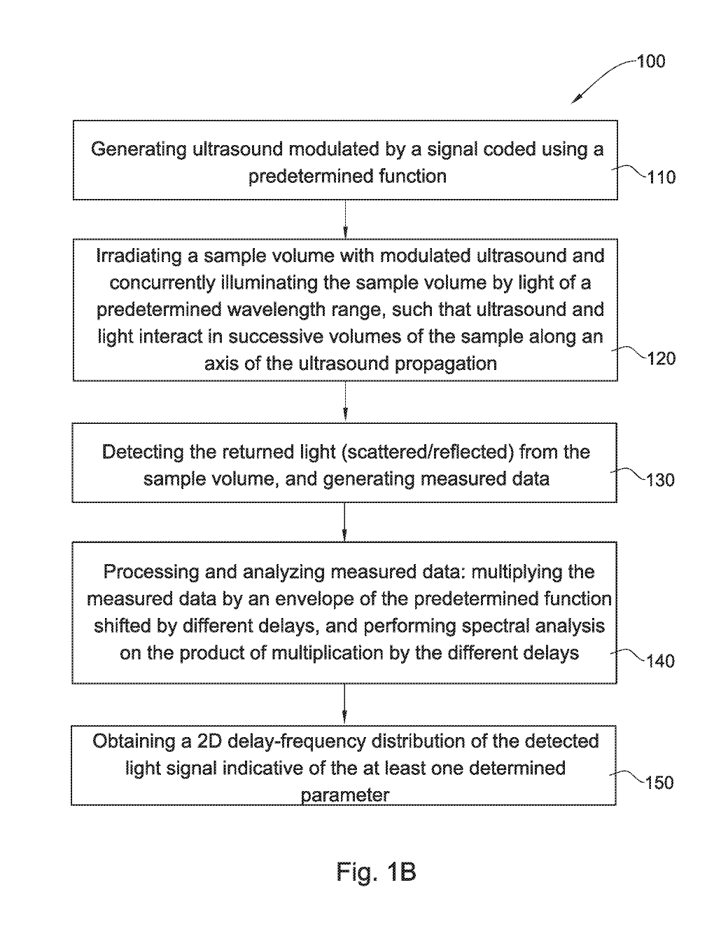

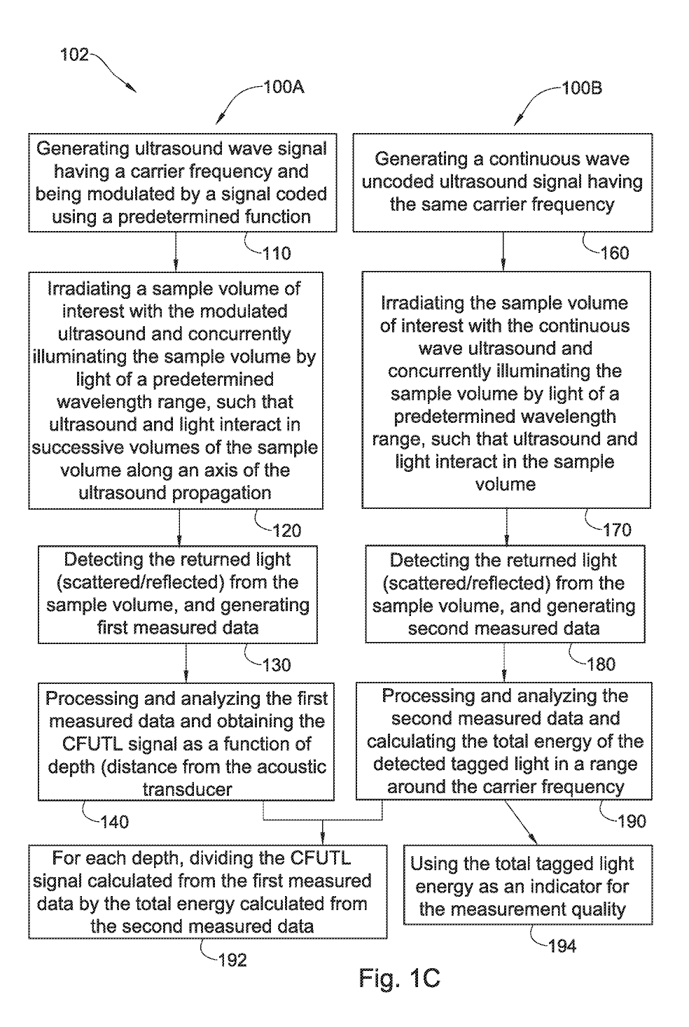

Referring to FIG. 1A, there is illustrated, by way of a block diagram, a measurement system 10 of the present invention configured and operable for characterizing a subject's tissue by its spectral data and determining one or more parameters of the subject. The system includes a control unit 12 which is configured as a computerized system including inter alia input/output utilities 12A, memory utility 12B, and a data processor and analyzer utility 12C which is configured and operable according to the invention for processing input measured data.

The measured data may be received from a measurement unit 14 in real time, i.e. during the measurement session in which case the control unit operates in a so-called on-line data processing mode, or from a storage device 15 (shown in dashed lines) in which the measured data has been previously stored and the control unit thus operates in an off-line processing mode. The control unit 12, or at least its data processor utility 12C, may be integral with the measurement unit 14 or with the storage device 15, or may be associated with a standalone unit/system connectable to the measured data source (measurement unit 14 or storage device 15) via wires or wireless signal communication, e.g. via a communication network. Hence, the control unit 12 is equipped/installed with an appropriate communication utility. The construction and operation of such communication utilities are known per se and do not form part of the present invention, and therefore need not be described in detail.

The control unit 12 may further include an illumination controller 12D configured and operable for communication with an illumination assembly associated with measurement unit 14. Such illumination assembly includes a light source unit 16A associated with one or more light output ports 14A. In the present example, the measurement unit 14 is configured as a probe to be brought closer to/in contact with a subject under measurements, and includes one or more light output ports (illumination ports) 14A optically coupled with an external/internal light source unit 16A, one or more light input ports (light collection ports) 14B optically coupled with an external/internal light detector 16B and forming together a detection assembly, and acoustic output port(s) 14C connected to external/internal acoustic wave generators 16C and 16D forming together a transducer assembly. The acoustic generators 16C and 16D actually present different functional utilities for respectively generating coded (e.g. pulsed or CW) and uncoded CW acoustic radiation, and may thus be implemented by a single acoustic generator unit operating in two modes of coded (pulsed) and continuous wave fashion, or as two separate generator units. It should be understood that light source and/or light detector and/or acoustic wave generator(s) may be integral with the measurement unit 14; as well as any or all of the light source, light detector and acoustic wave generator(s) may be integral with the control unit 12.

The measurement technique of the invention utilizes modulated acoustic signals in the form of a predetermined function of at least one parameter of the acoustic radiation which varies over time during a measurement session (measurement time interval). To this end, as further shown in the figure, a coded signal generator 12E is provided, being either a separate utility of the control unit 12 and connectable to the acoustic wave generator 16C, or being integral with the transducer assembly (e.g. integral with the acoustic wave generator).

In some embodiments, as will be further explained below, the measurement technique of the invention may utilize two measurement sessions carried out in a predetermined order: during a first measurement session, modulated acoustic signals in the form of a predetermined function of at least one parameter of the acoustic radiation which varies over time are transmitted via the acoustic generator 16C (or via a first mode of a one unit acoustic generator). To this end, as further shown in the figure, a coded signal generator 12E is provided, being either a separate utility of the control unit 12 and connectable to the acoustic wave generator 16C, or being integral with the transducer assembly (e.g. integral with the acoustic wave generator). During a second measurement session, continuous not modulated acoustic signals are transmitted via the acoustic generator 16D (or via a second mode of a one unit acoustic generator). It should be understood that the terms “first” and “second” are used only to distinguish between the measurement sessions which can be performed concurrently, given that the detected signals can be distinguished (e.g. by using two different carrier frequencies for the acoustic radiations), or sequentially in either order.

Reference is made to FIG. 1B illustrating a flow chart 100 exemplifying a method carried out by the above-described measurement system 10 utilizing the control unit 12 of the invention for characterizing a subject's tissue by its spectral data and determining one or more parameters of the subject. This flow chart exemplifies the system operation for generating measured data. Ultrasound modulated by a coded signal using a predetermined function is generated (step 110), and a sample volume of the tissue, such as a tissue in the body, is concurrently irradiated with the modulated ultrasound and illuminated by light of a predetermined wavelength range (step 120), such that ultrasound and light interact in successive volumes of the tissue along an axis of the ultrasound propagation.

As a non limiting example, the ultrasound is generated as a continuous wave to gain high signal to noise ratio (SNR). The aim of modulating the signal by the predetermined function is to enable determination of the source/location/depth from which a specific backscattered light signal arrived to a light detector. The control unit 12 generates a continuous signal modulated (coded) using the predetermined function. The ultrasound transducer receives the modulated continuous wave in the form of electrical generated coded signal and generates an ultrasound wave that is transmitted to the examined tissue. The light source and detector operate to illuminate the tissue region (at least part thereof) and detect a light response of the illuminated tissue which includes light tagged by the ultrasound.

Generally, under certain simplifying assumptions, the AC detected intensity of light modulated by an ultrasound wave may be described as:

I_{ac}(t)=Iₐᵣ·Re{e^{-i[ω}^{ᵤₛ}^{t+φ}^{ₐᵣ}^{]}} 1)

where, ωᵤₛ is Ultrasound frequency (Carrier frequency), φₐᵣ is an arbitrary phase shift, and Iₐᵣ is the amplitude. The spectral distribution of a modulation by a continuous wave (CW) signal is given by a Fourier integral:

[symbol]

_{ac}(ω)=∫ₜI_{ac}(t)·e^{-iωt}dt=Iₐᵣ·δ(ω−ωᵤₛ) 2)

In case the modulated signal also includes a random phase modulation (due to Brownian motion, flow etc.), an additional phase shift is present, namely:

I_{ac}(t)=Iₐᵣ·Re{e^{-i[ω}^{ᵤₛ}^{t+φ}^{ᵥ}^{(t)]}} 3)

with the spectral analysis yielding:

[symbol]

_{ac}(ω)=Iₐᵣ·δ(ω−ωᵤₛ)⊕Γᵥ(ω) 4)

where ⊕ stands for convolution, and Γᵥ(ω)=∫ₜ γᵥ(t)·e^{-iωt}dt is the Fourier transform of γᵥ(t)=e^{-iφ}^{ᵥ}^{(t)}.

For a set of volume elements vᵢ along the trajectory/axis of propagation, where each volume has its random phase modulation effect, the resulting detected light signal would be:

I_{ac}(t)=Re{Iₐᵣ·e^{-i[ω}^{ᵤₛ}^{t+Σ}^{ᵢ}^{φ}^{ᵥᵢ}^{(t)]}} 5)

with the resulting spectrum:

[symbol]

_{ac}(ω)=Iₐᵣ·δ(ω−ωᵤₛ)⊕Γₜₒₜₐₗ(ω) 6)

where:

Γₜₒₜₐₗ(ω)=Γ₁(ω)⊕Γ₂(ω)⊕ . . . ⊕Γᵢ(ω)⊕ . . . 7)

Thus, the overall spectral broadening is an accumulative result of many broadening processes.

In some embodiments of the invention, the predetermined function that modulates the continuous acoustic wave is a Golay code. Golay coding method can be used to effectively modulate only a specific volume, at a predetermined depth/distance from the transmitting plane, and would thus characterize the specific delay of acoustic radiation.

This Golay code may be implemented by transmission of ultrasound waves, with the following shape:

Golay(t)=Gₑₙᵥ(t)·Aᵤₛ cos [ωᵤₛt] 8)

As described above, the tissue is concurrently irradiated by such modulated ultrasound wave during a predetermined time interval and illuminated by light of a predetermined wavelength range, such that the ultrasound and light interact in successive volumes of the tissue along an axis of the ultrasound propagation. Scattered light tagged by ultrasound is detected and corresponding measured data is generated (step 130). The measured data is a coded signal indicative of a time function of the spectral intensity/profile of the detected light signal, where the time points (delays) correspond to successive locations inside the tissue along the general axis of ultrasound propagation.

If we assume that the moving scatterers are limited to a single plane at a distance R₁ from the transducer plane, the train of +1 and −1 in the Golay envelope Gₑₙᵥ(t) flips the phase of the intensity pattern Aᵤₛ on the detector which now becomes a Golay-coded intensity trace:

I_{Golay-coded}(t)=Re{Iₐᵣ·Gₑₙᵥ(t−τ_{R1})·e^{-i[ω}^{ᵤₛ}^{t+Σ}^{ᵢ}^{φ}^{ᵥᵢ}^{(t,τ}^{_{R1}}^{)]}} 9)

where τ_{R1}=R₁/Vᵤₛ is the time delay of the Golay train, at a distance R₁ from the transducer plane, and V_{u·s} is the Ultrasound velocity in the sample/tissue.

The measured data, in its digital representation, is processed and analyzed (step 140). The analysis may include multiplying the measured coded signal by an envelope of the predetermined function (the conjugated Golay code) shifted by different delays, and for each delay calculating the spectral data, e.g. performing a Fourier transform on the product of multiplication by the different delays. Alternatively, spectral filtering may be applied to the product of multiplication by the different delays. Thus, generally, “spectral processing” is performed, including calculation of spectral data as well as any other suitable spectral analysis such as spectral filtering.

Accordingly, the time-trace I_{Golay-coded}(t) is multiplied by Gₑₙᵥ(t−τ′) to obtain the Golay-decoded trace:

I_{Golay-decoded}(t)=I_{Golay-coded}(t)·Gₑₙᵥ(t−τ′)==Re{Iₐᵣ·Gₑₙᵥ(t−τ_{R1})·Gₑₙᵥ(t−τ′)e^{-[ω}^{ᵤₛ}^{t+Σ}^{ᵢ}^{φ}^{ᵥᵢ}^{(t,τ}^{_{R1}}^{)]}} 10)

which for τ′=τ_{R1}, becomes:

I_{Golay-decoded}(t,τ_{R1})=Re{Iₐᵣ·e^{-i[ω}^{ᵤₛ}^{t+Σ}^{ᵢ}^{φ}^{ᵥᵢ}^{(t,τ}^{_{R1}}^{)]}} 11)

It can be appreciated that I_{Golay-decoded} has a spectrum similar to that already seen in equation (6). In the case of many such time traces (or many delay times τ=R/Vᵤₛ) getting to the detector from many planes R, the total intensity Ĩ would be:

Ĩ_{Golay-coded}(t)=Re{∫_{Σ}Iₐᵣ·Gₑₙᵥ(t−τ)·e^{-i[ω}^{ᵤₛ}^{t+Σ}^{ᵢ}^{φ}^{ᵥᵢ}^{(t,τ}^{_{R}}^{)]}dt} 12)

Here Σᵢφᵥᵢ(t,τ′) is the phase modulation originating from the slab located at distance R=V_{usτ}·τ′.

Since the Golay code has the following property:

∫_{τ}Gₑₙᵥ(t−τ)·Gₑₙᵥ(t−τ′)·dT=δ_{ττ}, 13)

where δ_{ττ}, is Kronecker's delta, then signals arriving from other distances are expected to interfere destructively in the time trace. Thus, when the intensity time-trace is multiplied by a shifted Golay envelope Gₑₙᵥ(t−τ) and a Fourier integral is performed, the following is obtained:

Ĩ_{GD}(ω,τ)=Re{∫_{τ}∫_{τ}IₐᵣGₑₙᵥ(t−τ)Gₑₙᵥ(t−τ′)e^{-i[ω}^{ᵤₛ}^{t+Σ}^{ᵢ}^{φ}^{ᵥᵢ}^{(t,τ}^{_{R}}^{)]}e^{-iωt}dτdt} 14)

which, due to equation (13), becomes:

Ĩ_{GD}(ω,τ)=Iₐᵣ·δ(ω−ωᵤₛ)⊕Γₜₒₜₐₗ(ω,τ) 15)

where Γₜₒₜₐₗ(ω,τ) is the spectral shape/broadening resulting from photon trajectories going through a plane at a distance R=V_{usτ}·τ.

Thus, the delay-frequency distribution expressed by equation (15) is obtained (step 150), which describes the frequencies found at each delay/depth. The different frequencies are a measure of the moving centers at each depth, thus the more frequencies are present at the specific location (delay) the more variability is present with regards to moving centers at said location in the medium.

On the other hand, looking at a specific frequency, the distribution delivers information about the intensity in time, i.e. the intensity at the depth corresponding to the delay in time, of the signal possessing the specific frequency. As described in WO 2008/149342, assigned to the assignee of the present application, and as indicated above, the CFUTL (i.e. signal obtained by taking only the carrier frequency component of ultrasound calculated for each delay), is identical to the cross correlation between the coding function of the transmitted ultrasound and detected light signals. In fact, determining the distribution just at the carrier frequency rather than the full frequency distribution taking into account the medium induced effects on the acoustic radiation parameters (due to the movement of scatterers), for each delay, provides the cross-section of the 2D distribution along ω=ωᵤₛ. In other words, in case of ω=ωᵤₛ and substitute equation (10) in equation (14) the distribution becomes:

Ĩ_{GD}(ωᵤₛ,τ)=τ′t{IGolay-codedt}·Genvt−τ′·e−iωustdτ′dt=CFUTLτ 16)

As explained earlier, by using ultrasound tagging of light, it is possible to determine, amongst other things, the light distribution in the tissue and variations in blood flow within the measured volume. Because the ultrasound tagged light (UTL)) depends on the amplitude of light and the amplitude of acoustic pressure wave that is coupled to the tissue, there is a need to decouple the two parameters (light and acoustic energy), in order to determine optical properties of the tissue, such as color (oxygen saturation) and local blood flow effects.

One way to decouple the amplitude of the ultrasound, is by using several wavelengths of light, and divide the UTL profile obtained using the different wavelengths of light, one by the other (as described in WO 2008/149342). When only one wavelength of light is used, there is a need to decouple the effect of variability in the amplitude of the ultrasound waves that are coupled into the tissue on the obtained UTL light profile.

As described in U.S. Pat. No. 8,336,391, assigned to the assignee of the present application, a blood flow index (CFI) can be calculated by dividing the average, or “direct current” (DC) light intensity by the average CFUTL value in a certain interest range (IR) along the time/position axis. However, the energy parameter combines both the effect of the light intensity (DC) and that of the ultrasound amplitude. Thus, it essentially provides more data, and eliminates the dependency of CFI on the ultrasound coupling in particular, and on the ultrasound power transmitted to the subject's superficial tissue in general. In one embodiment of the present invention decoupling is obtained by dividing an energy parameter by the amplitude of the CFUTL signal (defined as the cross correlation between the detected light signal and the coding function of the transmitted ultrasound signal (as defined by CCA(λ,μ) in WO 2008/149342), or the opposite way around. Furthermore, while the DC light intensity conveys information regarding the light coupling to the examined tissue, the total tagged light energy also additionally conveys information regarding the ultrasound coupling to the tissue, enabling improved monitoring of the measurement quality and indication of sub-optimal coupling conditions, that can be used online or offline.

Reference is now made to FIG. 1C, illustrating a flow chart 102 exemplifying another method that may be carried out by the above-described measurement system 10 utilizing the control unit 12 of the invention for characterizing a subject's tissue by the detected light data and determining one or more parameters of the subject. This flow chart exemplifies the system operation for generating first and second measured data in two separate measurement sessions 100A and 100B. It should be understood that the measurement session 100A is includes the same measurement steps obtained in the method described in FIG. 1B, and what is referred to herein as “first measured data” is the same as the measured data obtained in applying the method described in FIG. 1B.

It should also be noted that the measurement sessions 100A and 100B can be performed in any order, i.e. session 100A followed by session 100B or vice versa. In the session 100A, the CFUTL signal for each depth (location/delay) is obtained considering the carrier frequency of the coded acoustic radiation. However, it should be noted that more general spectral information may be extracted from the first measured data as described above. In the measurement session 100B, utilizing the uncoded CW acoustic radiation, the second measured data can be used for calculating the total energy of the tagged portion of the detected light from the entire region of interest. Then, division of the CFUTL for each depth from session 100A by the total energy from session 100B, illustrated in step 192, results in normalized figures of the light parameters obtained. This normalization mitigates the influence of the ultrasound source and light source variability, as well as the coupling conditions, on the detected light, which means that the measurements are independent of various conditions affecting the results and thus are more accurate and uniform and comparable across examined subjects. It further means that variations in the measurement quality due to the ultrasound source, light source and coupling conditions' variability can be continuously monitored, by using the total tagged light energy as an indicator of the measurement quality.

Accordingly, in measurement session 100B, an uncoded continuous wave of ultrasound is generated (step 160) and irradiated towards the same tissue volume which was irradiated during session 100A. Concurrently, the tissue volume is illuminated with light of a predetermined wavelength range (step 170). The backscattered light is detected forming a second measured data (step 180). The second measured data is processed such that the tagged light is extracted and analyzed in the spectral domain to calculate the total energy of the detected tagged light in a frequency range around the carrier frequency (step 190). To this end, any known suitable spectral analysis technique can be used. The overall energy is equivalent to the integral of the power spectra calculated at each delay, in a predefined bandwidth bw around the carrier frequency.

For example, the integral is calculated for frequencies from 0.5 times the carrier frequency to 1.5 times the carrier frequency, or any other predetermined range, or alternatively a dynamically determined range, that can account for additional factors, such as noise.

The last stage according to the method of the invention includes two independent steps. The first step is dividing the CFUTL signal for each depth along the monitored volume, obtained in step 140, by the total energy parameter, obtained in step 190 (step 192). The resulting figure for each depth/location is actually a normalized value of the CFUTL. This enables comparing the CFUTL values obtained at different depths/locations during the same or different measurements for the same subject or for different subjects. This normalization mitigates uncontrolled variability introduced due to the coupling of ultrasound to the examined volume resulting in accurate tissue light properties. The second step (step 194) is using the total energy parameter obtained in step 190 as an indication for signal quality due to acoustic coupling, allowing the acoustic coupling repair when needed.

The inventors have conducted a preliminary feasibility experiment relating to the light energy parameter and the ultrasound radiation. The results of the experiment prove that the energy parameter is dependent on the ultrasound amplitude, but independent on the flow, and thus provide a feasibility proof of the use of the energy parameter as an elimination factor for the UTL dependency on the ultrasound coupling.

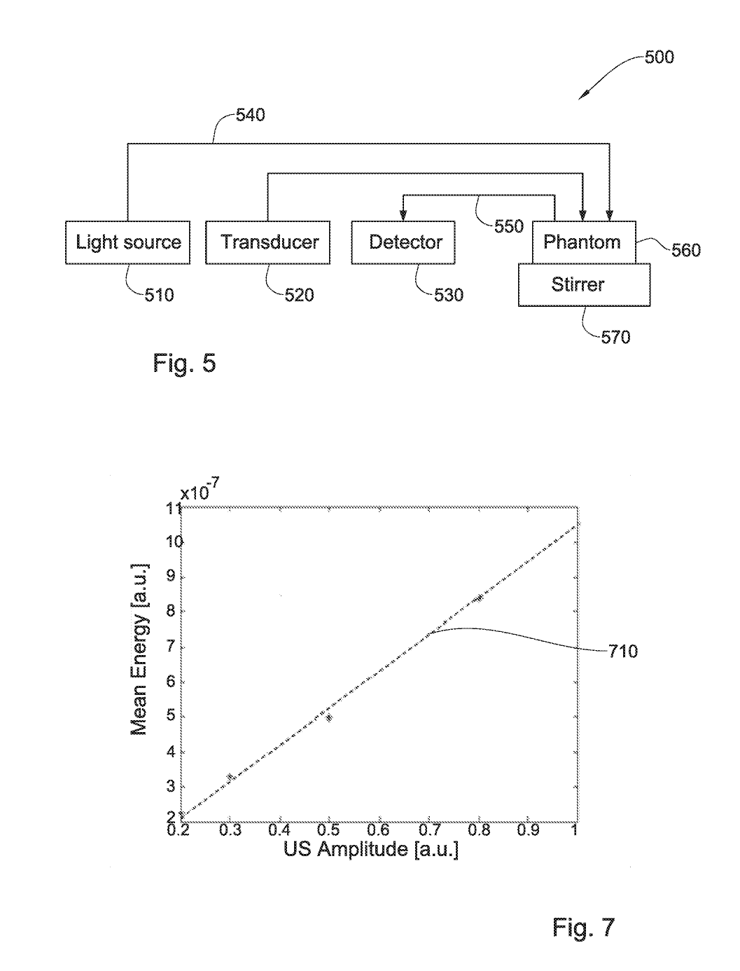

FIG. 5 illustrates the experimental setup 500 used. Light from a long coherence length (>1 m), 830 nm wavelength laser diode 510 (constituting a light source) was coupled into a 62.5 μm multi mode fiber 540, whose light output port is at a phantom 560 containing Glycerol+TiO2. The phantom 560 was placed on a stirring plate 570. A 0.995 MHz ultrasound was generated by acoustic radiation generator and transducer assembly 520 and transmitted into the phantom 560, with the light simultaneously. It should be noted that the acoustic transducer (its output port) may have a ring-like geometry, and the light output port of the illuminating fiber 540 may be arranged concentrically with the acoustic port (center illumination configuration). Another 62.5 μm multi mode fiber 550 was inserted to the phantom 560, approximately 11 mm away from the transmission fiber 540. This receiving fiber 550 collected light from the phantom 560 and redirected it towards an avalanche photodiode 530 (APD).

In order to create two different states, the stirring plate 570 was used along with a magnet (not shown) which was placed at the bottom of the phantom 560. The first state, in which the stirring plate 570 was “off”, was a “no flow” state. The second state was a “flow” state, in which the plate 570 was “on” and rotated the magnet at the bottom of the phantom 560 to generate movement of the optical scatterers within the phantom. In both states, the power spectrum of the light intensity was calculated and analyzed. This procedure was repeated for several ultrasound amplitudes.

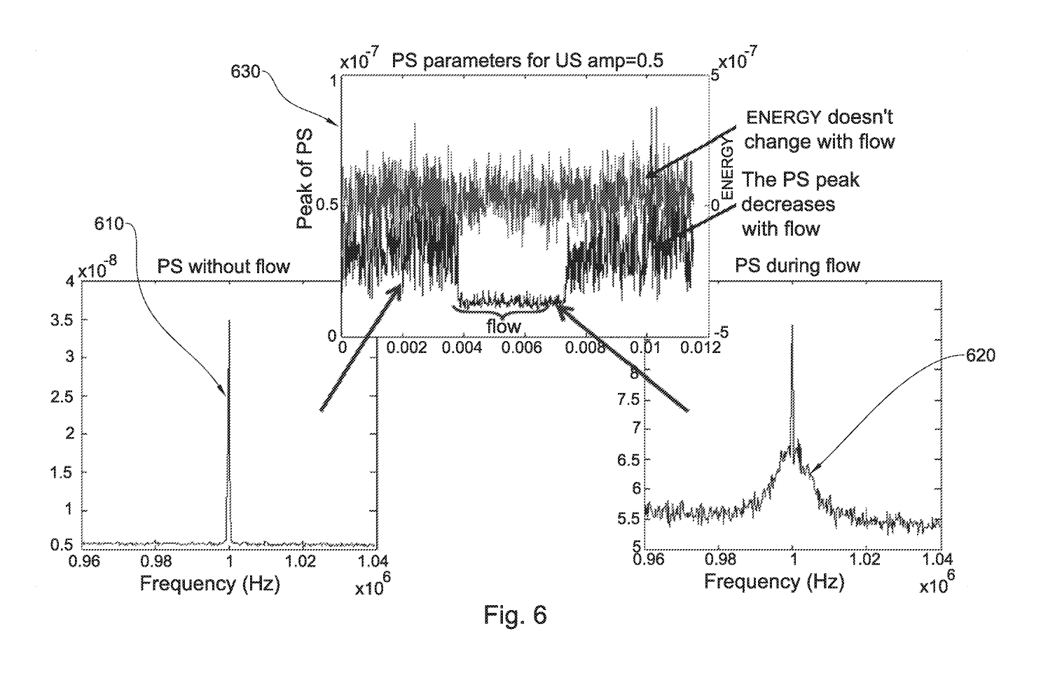

FIG. 6 shows the experimental results. Line 610 is the power spectrum in the first state, when there was no flow, and line 620 is the power spectrum in the second state, when the flow was present. It can be seen in the upper middle graph 630 that the energy parameter does not vary when the flow is varied, while the power spectrum peak decreases significantly with flow. This was repeated at several amplitudes for the ultrasound, in order to simulate different coupling conditions.

FIG. 7 demonstrates the dependence of the energy parameter on the ultrasound amplitude. The graph includes the Mean energy, the Y-axis, calculated throughout the feasibility experiment in different ultrasound amplitudes, the X-axis. A linear relation 710 is observed and apparent.

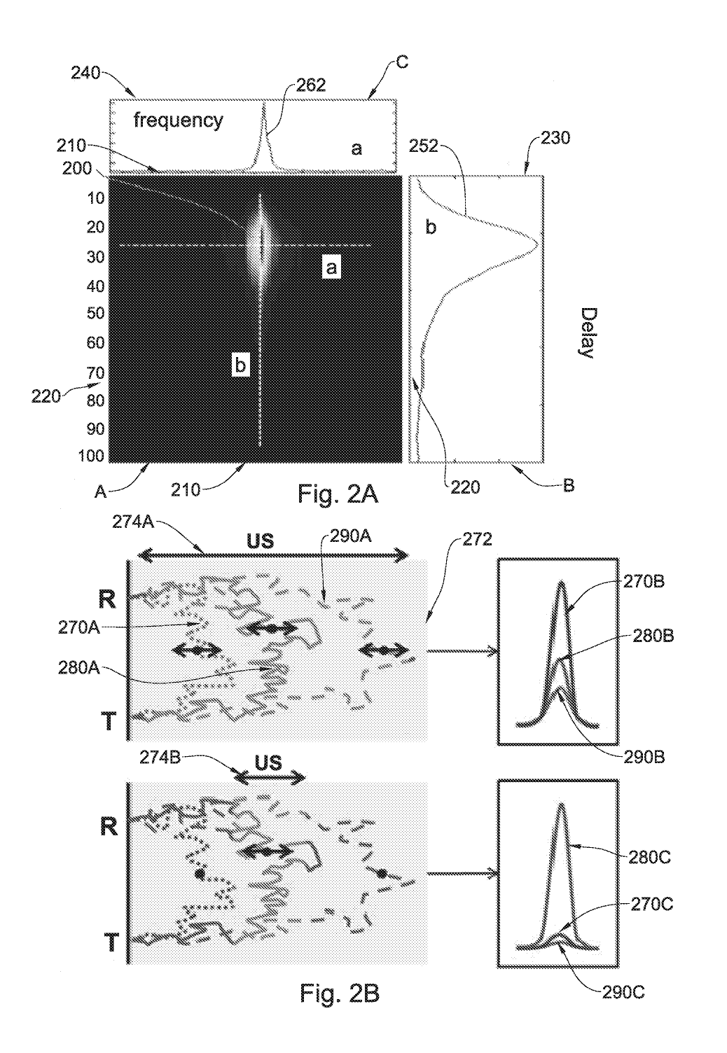

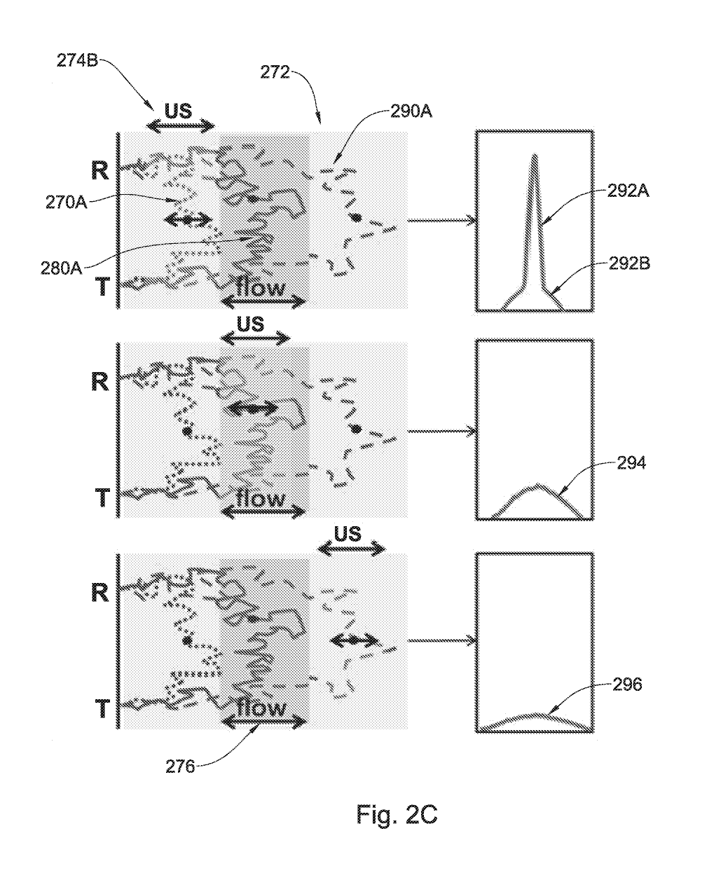

As said earlier, the processing of the detected light data measured in session 100A (referred to as the first measured data in FIG. 1C, or the measured data in FIG. 1B) may provide spectral information of plurality of frequencies at each distance/delay from the transmitting plane. This will be described in more details below in connection with FIGS. 2-4.

Reference is made to FIG. 2A which is a graphical representation exemplifying a delay-frequency distribution 200 obtained according to the present invention from actual measurement made on a human head. As shown in part A of the figure, the horizontal axis 210 is a frequency axis and the vertical axis 220 is a delay (depth/position) axis.

The vertical dotted cross-section b at ω=ωᵤₛ yields the time trace of the light intensity, described previously in WO 2008/149342 and known as CCA or CFUTL. This is shown in part B in which a graph, having the delay 220 at one axis and the signal intensity 230 at a second axis, corresponds to the CFUTL graph 252.

The horizontal dashed cross section a yields spectral information at a specific depth. This is shown in part C in which a graph, having the frequency 210 at one axis and the signal intensity 240 at a second axis, corresponds to the spectral distribution 262.

Reference is made to FIG. 2B showing simulation for concurrent illumination and irradiation of a region of interest 272 with electromagnetic radiation and ultrasound. Different light paths 270A, 280A and 280C explore different depths (volumes) inside the region of interest. Most probable paths are such that make a relatively short “banana-like” shape (270A). As paths get longer, their probability of reaching the detector gets smaller (280A) and smaller (290A). Black points on those paths designate typical scattering sites. Here, T and R stand for transmission and reception, respectively. In the example illustrated in the upper part of the figure, the whole region of interest is excited by ultrasound 274A. The spectral shapes for the three paths 270A, 280A and 290A may generally look like the curves 270B, 280B and 290B respectively. As seen, the closer path to the source (i.e. 270A) which is the most probable to be detected is dominant. In the example illustrated in the lower part of the figure, only part of the region of interest is excited by ultrasound 274B. In this case, the ultrasound 274B modulates only a volume overlapping mainly with the middle path (280A), and thus the spectrum is dominated by this path, i.e. curve 280C. The shorter path 270A is almost not modulated while the longer path 290A is partially modulated. Both paths 270A and 290A are less expressed as shown by curves 270C and 290C due to no modulation in the case of path 270A, or due to their relatively smaller probability for detection and smaller ultrasound overlap (i e smaller interaction with ultrasound and thus incomplete modulation) as the case with path 290A.

FIG. 2C illustrates a situation in which only part of the region that contains a flowing medium 276 is of interest. The ultrasound propagates through the region 272 and excites different parts at different time delays. In the upper panel of the figure, ultrasound excites a part containing the path 270A, which is upstream of the region of interest 276 with respect to the direction of ultrasound propagation. In this situation, the detected light spectrum (on the right) is mainly affected by a carrier frequency of the ultrasound interacting with light along trajectory 270A, which is not broadened/affected by ultrasound and light interaction at the flowing medium, as shown by curve 292A, and only some broadening occurs as shown by sides 292B, due to the detection of light returned from path 280A and being thus partially modulated by the ultrasound before and after passing through the region of flow 276. In the middle panel, the part excited by ultrasound 274B overlaps with the flow volume 276, thus at the “modulated trajectories” (280A) the detected spectrum, is affected by interaction with ultrasound at the flow medium/volume and the expected spectrum will be similar to curve 294. Turning to the lower panel, the ultrasound 274B excites a layer/volume which is downstream of and outside the flow volume 276, but, although the total number of photons travelling along the path 290A is relatively small (because long path means less probability to get back to the detector), and accordingly the intensity (amplitude of spectrum) is small, the spectrum 296 is also broadened, as the photons in this trajectory 290A pass through the flow volume 276 as well.

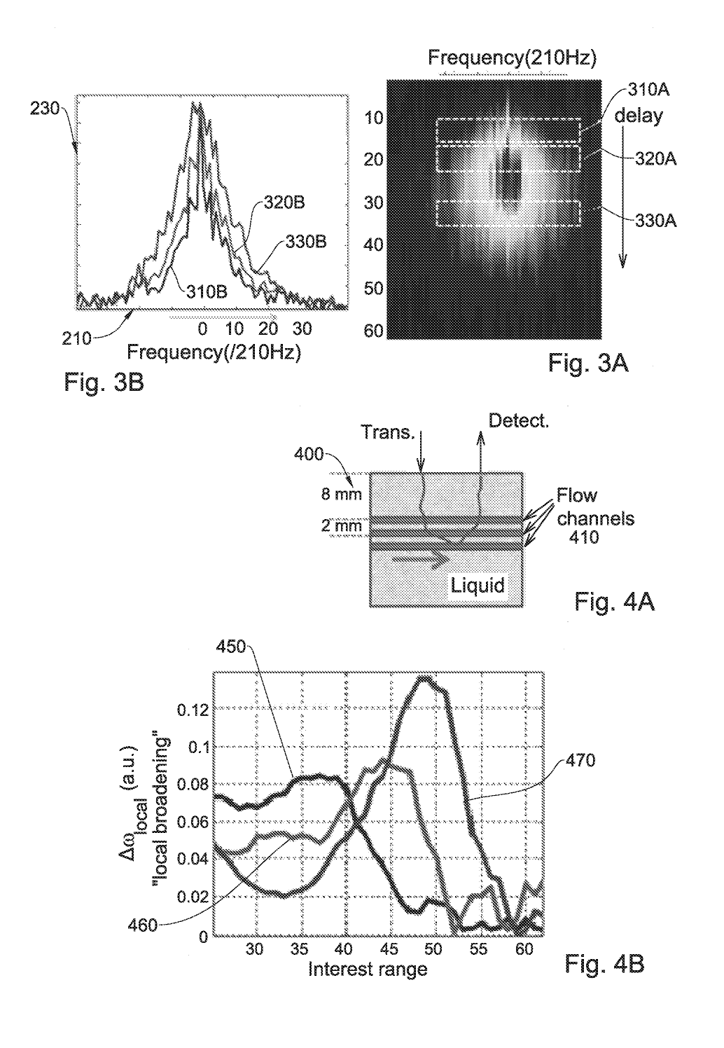

Referring to FIGS. 3A and 3B there are presented results obtained from a liquid phantom in which the liquid contains stirred scattering centers. Measurements have been done at three slabs 310A, 320A and 330A of different depths/distances from the ultrasound and light sources. More specifically, slab 310A is located in the pixel range of 10-15 deep, slab 320A is in the pixel range of 18-22 deep and slab 320A is in the pixel range of 30-35 deep, where each pixel is roughly equivalent to a depth of 0.4 mm. FIG. 3B shows three spectrum graphs at three different depths, 310B, 320B and 330B which are the spectrum graphs at slabs 310A, 320A and 330A respectively. As seen in FIG. 3B, for deeper slabs/planes (larger delay times), the spectral width is larger. This effect is expected because as stated above, broadening is accumulated as light gets deeper into tissue. Moreover, by subtracting spectral width measured for a given depth R, from that measured at more distant depth (R+dR), the amount of broadening contributed specifically by the deeper layer can be deduced. Thus, quantitative tissue flow-cross-section or profile may be obtained.

A possible realization of spectral width quantification of Ĩ_{GD}(ω,τ) at a given delay may be calculated as the ratio:

$${Δω}_{GD} ( τ ) = \frac{{\tilde{I}}_{GD} ( {ω}_{bw} , τ )}{{\tilde{I}}_{GD} ( {ω}_{us} , τ )} \qquad 17 )$$

where Ĩ_{GD} (ω_{bw},τ) is an energy at a given spectral bandwidth in the vicinity of ωᵤₛ, bw. This energy can be calculated directly by performing a Fourier transform to obtain Ĩ_{GD} (ω,τ) and then summing over the frequencies within bw, for example (when bw is symmetric around ωᵤₛ):

$${Δω}_{GD} ( τ ) = \frac{{\sum }_{- {ω}_{\frac{bw}{2}}}^{{ω}_{\frac{bw}{2}}} {\tilde{I}}_{GD} ( {ω}_{us + i} , τ )}{{\tilde{I}}_{GD} ( {ω}_{us} , τ )} . \qquad 18 )$$

However, it may be in some cases preferable (e.g. for reduction of computational load) to directly calculate the bandwidth energy instead of sum

$${\sum }_{- {ω}_{\frac{bw}{2}}}^{{ω}_{\frac{bw}{2}}} {\tilde{I}}_{GD} ( {ω}_{{ω}_{us + i}} , τ ) ,$$

by means of spectral-domain filtering, e.g. using an effective bandwidth IIR filter such as a bi-quadratic filter.

Reference is now made to FIGS. 4A and 4B, showing in FIG. 4A a schematic diagram of a liquid channel phantom 400 used for creating and recording a different signal from different depths. A scattering fluid is injected at channels 410 at each depth separately. The first shallowest channel is at 8 mm far from the ultrasound source, the second middle channel is at 10 mm far and the third deepest channel is 12 mm far from the ultrasound source.

FIG. 4B shows the differential spectral broadening obtained by subtracting the spectral width measured at a given delay depth/distance from the spectral width at a longer distance (or higher time delay) for the flow phantom 400, basically calculating the derivative of the width function relative to the time delay. The line 450 represents a trace of the above defined differential broadening at a distance of 8 mm below the ultrasound transducer, i.e. the line represents a difference between the spectral width obtained at depth 8 mm and the spectral width obtained just above 8 mm (at a slightly shallower location). Since both signals contain accumulation of all the broadening effects until that point, the difference between the two signals is calculated to acquire the effects at depth 8 mm. Similarly, the lines 460 and 470 represent differential broadening at 10 mm and 12 mm below the ultrasound transducer, respectively, as they represent difference between signal at each depth and the signal up to that specific depth. The positive slopes in each of the traces 450, 460 & 470, are indicative of the beginning of an injection of liquid into the channels 8, 10 & 12 mm, respectively, and the negative slopes are indicative of the stopping of liquid injection, respectively.

The inventors of the present invention have conducted two further experiments to validate some of the features of the present invention. The first experiment was aimed to verify a linear relation between the total energy parameter and the detected DC light intensity that consists of untagged light, and the second experiment was aimed to verify correlation (linearity) between the total energy parameter and the ultrasound tagging radiation amplitude. Both experiments were conducted on a subject's forehead using a system constructed according to the invention.

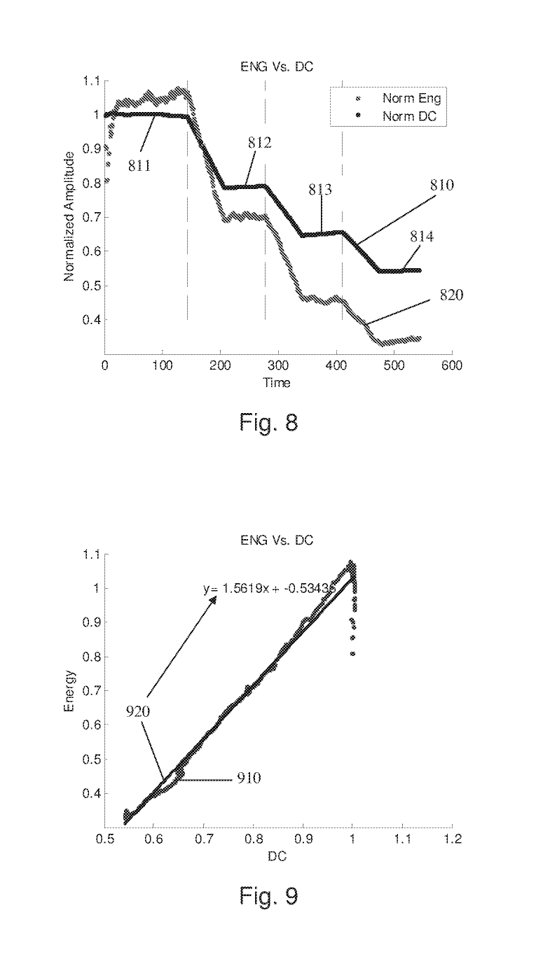

Reference is made to FIGS. 8 and 9 showing results of the first experiment.

During this experiment, the illuminating light radiation was decreased gradually resulting in that the detected light intensity measured by the detector decreased gradually, while the ultrasound amplitude and coupling were kept constant. The light transmitting optic fiber was connected to the control unit of the system via an attenuator which enabled control on the transmitted light power. FIG. 8 shows a plot of normalized amplitude values of each of the detected DC light intensity 810 and the normalized total light energy 820 against time. FIG. 9 shows a plot of the total energy of the detected light 910 against the tagged light intensity 920. A continuous measurement of twenty minutes was recorded. Every five minutes the attenuator was re-set to enable transmission of less light, thus the detected light intensity was decreased accordingly creating four different light intensity levels 811, 812, 813 and 814. The tagged light signal was recorded and the total energy parameter was calculated as explained before. As clearly shown in FIG. 8, the behavior of light intensity 810 and total energy parameter 820 is similar as would be expected. Also it is clear from FIG. 9 that plotting the normalized total energy versus the normalized light intensity (line 910) reveals a distinct linear relation 920 between the two parameters.

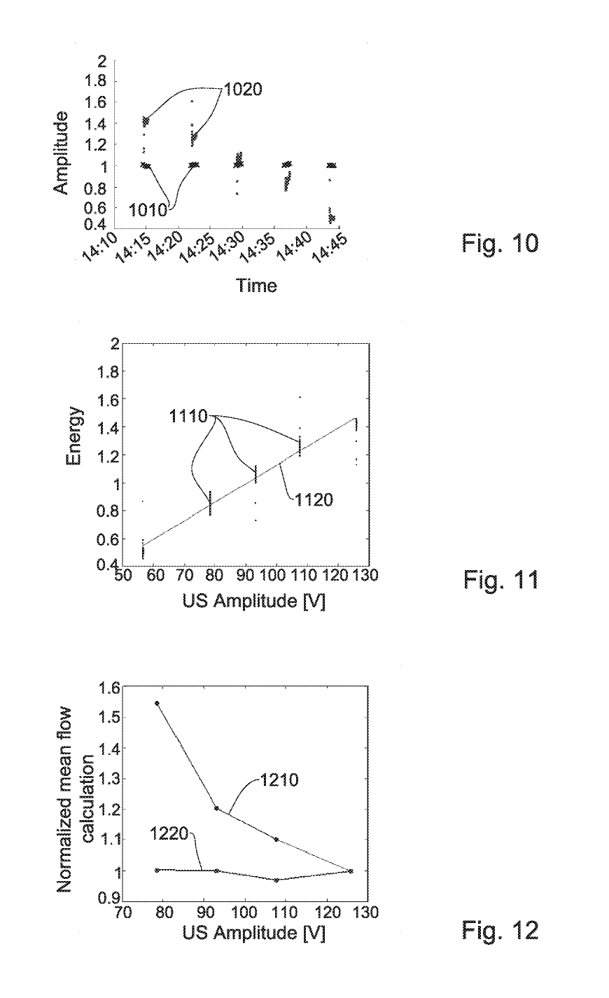

Reference is made to FIGS. 10 and 11 showing results of the second experiment. During the second experiment, as shown in FIG. 10, the light intensity was kept constant. In order to model different ultrasound coupling conditions, the ultrasound amplitude was set five times (for five different amplitudes) through the control unit of the system. FIG. 10 is a plot of amplitude values of each of the normalized detected DC light intensity 1010 and the normalized total light energy 1020 against time, while the tagging ultrasound's amplitude was changed. It is apparent from the figure that the transmitted light intensity was kept constant, as reflected in the normalized DC intensity, however the total energy was significantly changed, implying for its dependency on the US amplitude. FIG. 11 shows a plot of the detected total light energy against the ultrasound amplitude (points 1110). The figure illustrates the linear relation (line 1120) between the total energy parameter and the ultrasound amplitude. Thus, the total energy parameter can serve as an indicator for the measurement quality, i.e. the coupling of the ultrasound and electromagnetic radiation to the subject under examination. Further, a flow index (FI) was calculated in two different ways. This is shown in FIG. 12 illustrating a relation between normalized mean flow calculation (FI) and different ultrasound amplitudes (mimicking different acoustic coupling conditions). The first way that the FI was calculated was by normalizing the CFUTL with DC light intensity (line 1210), and the second way was by normalizing the CFUTL with the total energy parameter (line 1220). Between the two normalization methods, the results clearly show that the CFUTL normalization by the total energy parameter diminishes FI dependency on US amplitude.

Thus, the present invention provides a novel effective non-invasive technique for characterizing the properties of tissues/media. Turning back to FIG. 1A, the control unit 12 received measured data which has been continuously collected by a light detector during a certain time interval (measurement session), or two time intervals (two measurement sessions, as described with regards to FIG. 1C. The measured data collected during the first measurement session, in its digital representation, is processed by the data processor and analyzer utility 12C. The data processor and analyzer utility 12C comprises a decoder module 12G and a spectral processor module 12H (software/hardware). The decoder 12G utilizes data indicative of the predetermined coding function (e.g. receives this data from memory utility) used for modulation of ultrasound and data indicative of the ultrasound carrier frequency, and multiplies the measured data by an envelope of the coding function shifted at different delays of the acoustic radiation. The spectral processor module 12H applies a frequency-domain related analysis/filtering, to the product of multiplication, resulting in processed spectral data. This frequency-related analysis may be a Fourier transform, thereby obtaining a delay-frequency distribution, which is actually a position-related spectral data through the tissue depth. The analysis may also be application of spectral filters resulting in a local (delay-specific) or total estimation of spectral width. This spectral data is further processed by software module 12I for determining at least one parameter of the region of interest. This processing may include for example a calculation of local energy of light parameters and/or a total energy of light parameter, a calculation of the intensity distribution around the carrier frequency, and a calculation that utilizes results from previous calculation steps. The processing in module 12I may also include processing of local or total spectral width to deduce parameters such as the tissue's characteristic optical de-correlation time, and/or other parameters indicative of flow.

In the case of the two measurement sessions, according to FIG. 1C, the second measured data collected during the second measurement session (i.e. for uncoded CW acoustic radiation), is processed by the spectral processor module 12H to obtain the energy power spectrum of all the tagged light in the detected signal. The software module 12I is configured to calculate the overall tagged light energy in a predetermined frequency range around the carrier frequency of the continuous wave acoustic radiation. The software module 12I is also configured to divide the UTL amplitude by the overall energy, thereby yielding depth-specific tissue characteristics/parameters. Furthermore, the software module 12I is also configured to assess acoustic coupling quality (the measurement quality) by utilizing the total energy of the detected tagged light which teaches, as described earlier, about the acoustic coupling. If a non-expected change occurs, i.e. the total energy changes while the output of the acoustic radiation has not been changed, then this would indicate a change in the acoustic radiation coupling. The software module 12I may then send this information to a quality indicator utility 12J, which indicates and alerts in real time to the user about the change in the acoustic coupling.

1. A measurement system for use in determining at least one parameter of a subject, said system comprising:
(a) an acoustic device adapted for generating acoustic tagging radiation and for irradiating a region of interest of the subject with said acoustic tagging radiation propagating with a general propagation direction, said acoustic tagging radiation comprising modulated acoustic radiation in the form of acoustic wave having a carrier frequency and being modulated by a predetermined coding function of at least one parameter of the acoustic tagging radiation varying over time;
(b) an optical device adapted for illuminating the region of interest with electromagnetic radiation of at least one predetermined frequency, detecting an electromagnetic radiation response of the region of interest, and generating measured data corresponding to the detected electromagnetic radiation response; said optical device being operable concurrently with the acoustic device during at least a first measurement session, the measured data being thereby indicative of the electromagnetic radiation response to interaction between the acoustic tagging radiation and the electromagnetic radiation at successive positions in the region of interest along said general propagation direction during said at least first measurement session, the successive positions corresponding to successive delays of the interaction between the acoustic tagging radiation and the electromagnetic radiation during said at least first measurement session, and
(c) a control unit adapted for processing the measured data in both time and frequency domains and determining at least first data comprising spectral data as a function of position within the region of interest along said general propagation direction of the acoustic tagging radiation through the region of interest, such that each specific position of the successive positions in the region of interest is characterized by its spectral data comprising data indicative of tissue optical properties at said specific position.

2. The measurement system according to claim 1, wherein said processing of the measured data, by said control unit, comprises multiplying the measured data by an envelope of said predetermined coding function shifted at different delays of the acoustic tagging radiation, and applying spectral processing to product of the multiplication, thereby obtaining a delay-frequency distribution of the electromagnetic response being indicative of the at least one parameter of the region of interest.

3. (canceled)

4. The measurement system according to claim 2, wherein said spectral processing, by the control unit, comprises Fourier transform.

5. The measurement system according to claim 2, wherein said spectral processing, by the control unit, comprises spectral filtering.

6. The measurement system according to claim 1, wherein said processing of the measured data, by the control unit, comprises applying spectral analysis to spectral data from the successive positions along the trajectory of propagation of the electromagnetic radiation, thereby determining localized spectral broadening data of specific positions.

7. The measurement system according to claim 6, wherein said spectral analysis comprises determining a linear combination of the spectral data from the successive positions along the trajectory of propagation of the electromagnetic radiation.

8. The measurement system according to claim 6, wherein said spectral analysis comprises subtracting spectral data of first and second successive positions along the trajectory of propagation of the electromagnetic radiation, thereby determining localized spectral broadening data of the second position.

9. The measurement system according to claim 1, wherein said processing of the measured data, by the control unit, further comprises calculating a flow-depth distribution with absolute units.

10. The measurement system according to claim 9, wherein said calculating comprises determining a parameter of a profile of the spectral data in one or more of the successive positions along the trajectory of propagation of the electromagnetic radiation.

11. The measurement system according to claim 9, wherein said calculating comprises determining a width parameter of at least one peak in the spectral data in one or more of the successive positions along the trajectory of propagation of the electromagnetic radiation.

12. The measurement system according to claim 9, wherein said calculating comprises dividing a light energy parameter of said detected electromagnetic radiation by amplitude of a cross correlation between said coding function of the tagging acoustic radiation and said detected electromagnetic radiation response.

13. The measurement system according to claim 12, wherein said light energy parameter comprises the light energy in a spectral band around said carrier frequency in one specific position in the region of interest.

14. The measurement system according to claim 12, wherein said light energy parameter comprises an average of light energies in a spectral band around said carrier frequency in a plurality of positions in the region of interest.

15. The measurement system of claim 12, wherein said light energy parameter comprises a vector of light energies in a spectral band around said carrier frequency in at least two positions in the region of interest.

16. The measurement system according to claim 1, wherein said processing of the measured data, by the control unit, further comprises calculating a calibrated Calculated Flow Index (cCFI), being a function of the spectral data.

17. The measurement system according to claim 16, wherein said calculation comprises determining a width parameter of at least one peak in the spectral data in one or more of the successive positions along the trajectory of propagation of the electromagnetic radiation.

18. The measurement system according to claim 16, wherein said calculation comprises dividing a total energy parameter of said detected electromagnetic radiation by amplitude of a cross correlation between said coding function of the tagging acoustic radiation and said detected electromagnetic radiation.

19. The measurement system according to claim 18, wherein said processing comprises obtaining a local energy parameter for each delay by integrating power spectrum calculated at that delay along a frequency axis, and determining said total energy parameter as a sum of all said local energy parameters.

20. (canceled)

21. The measurement system according to claim 1, wherein said acoustic device is further adapted for generating acoustic tagging radiation in the form of a continuous uncoded acoustic wave having a second carrier frequency to propagate along said general propagation direction, thereby causing interaction between the continuous acoustic radiation and the electromagnetic radiation at the region of interest, said measured data further comprising second measured data indicative of detected electromagnetic radiation response from the region of interest to said interaction with the continuous acoustic radiation; said control unit being adapted for processing said second measured data and determining second data comprising spectral data of the region of interest, and utilizing at least one of the first and second data for determining a total energy parameter of the tagged portion of the detected electromagnetic radiation in a predetermined frequency range around the second carrier frequency.

22. (canceled)

23. The measurement system according to claim 21, wherein the generation of said acoustic tagging radiation in the form of the continuous uncoded acoustic wave of the second carrier frequency and detection of the electromagnetic radiation response to the interaction with said continuous uncoded acoustic wave is performed during a second measurement session.

24. (canceled)

25. The measurement system according to claim 21, wherein said processing of said measured data, by the control unit, comprises processing the first data and calculating a carrier frequency ultrasound tagged light (CFUTL) signal as a cross correlation between said predetermined coding function of at least one parameter and said electromagnetic radiation response.

26. The measurement system according to claim 25, wherein said processing further comprising dividing said CFUTL signal by said total energy parameter.

27. The measurement system according to claim 21, wherein said processing of said second data comprises calculation of the spectral width of said second data.

28. The measurement system according to claim 21, wherein said processing of said second data comprises determining Fourier transform of said second data.

29. The measurement system according to claim 21, wherein said spectral processing of second data comprises applying spectral filtering to said second measured data.

30. The measurement system according to claim 21, wherein said determination of said total energy parameter comprises utilizing the first data and obtaining a local energy parameter for each delay by integrating power spectrum calculated at that delay along a frequency axis, and determining said total energy parameter as a sum of all said local energy parameters.

31-41. (canceled)

42. A system for use in determining one or more parameters of a subject, said system comprising;
(a) an optical device configured for illuminating a region of interest with electromagnetic radiation of a predetermined frequency range, and for detecting an electromagnetic radiation response from said region of interest, and for generating measured data indicative of the detected electromagnetic radiation response;
(b) an acoustic device configured for irradiating said region of interest, while being illuminated, with first and second acoustic radiations propagating with a general propagation direction during respective first and second measurement sessions, wherein: the first acoustic radiation comprises acoustic tagging radiation in the form of acoustic wave having a first carrier frequency and modulated by a predetermined coding function of at least one parameter of the first acoustic tagging radiation varying over time, the second acoustic radiation comprising acoustic tagging radiation in the form of a continuous uncoded acoustic wave having a second carrier frequency, the measured data thereby comprising first and second data indicative of first and second interactions between the electromagnetic radiation with respectively first and second acoustic tagging radiations within the region of interest and the electromagnetic radiation at successive positions of the region of interest during the first and second measurement sessions; and
(c) a control unit configured and operable to process the first and second data, said processing comprising: determining first spectral data indicative of first electromagnetic radiation response from successive positions of the region of interest corresponding to successive delays of the interaction between the first acoustic tagging radiation and the electromagnetic radiation during said first measurement session, and second spectral data indicative of second electromagnetic radiation response of the region of interest and a total energy parameter of tagged portion of electromagnetic radiation around the second carrier frequency.
